(19) United States
(12) Patent Application Publication
(10) Pub. No.: US 2005/0074503 A1
(43) Pub. Date: Apr. 7, 2005

(54) Bilious balsam

(76) Inventor: Yakov Yevseyevich Brilman, Voorhees, NJ (US)

Correspondence Address: Yakov Brilman, apt. 508, 219 Laurel Rd., Voorhees, NJ, 08043, US

(21) Appl. No.: 10/742,527

(22) Filed: Oct. 5, 2003

(51) Int. Cl. A61K 33/24; A61K 33/00

(52) U.S. Cl. 424/650; 424/717

(57) ABSTRACT

For prevention of HIV/AIDS diseases, gripe and flu I am suggesting apply the solution “B” (Brilman's solution) including Pantocidi (Pantosept) −0.2%; Sodium chloridi 4%; Sodium hydrocarbonate 5%, and Sodium tetraborate 0.5% in distilled water. Partial disinfection is reached by using this solution and it is also used for cleaning the skin and mucous membranes including the mouth, sexual organs, anus, crotch, and also any infected material by use of mouthwashes, washing and irrigation. After washing with this solution I suggest applying externally the unguentum (cream or gel) “Oxopant” containing 0.2% Pantocidi (Pantosept and 0.5% Oxolini. This medicine is put on the skin and mucous membranes of the sexual organs, anus and crotch for 15-20 min. This preparation can also be successfully used for the prevention of gripe and flu viral infections with use of the mouthwash of the throat and drop in nose of the solution “B” (Brilman's solution), After apply “Oxopant” to mucous membranes of the nose.

Efficient says to fight the quickly spreading dangerous viral disease HIV/AIDS infection are unknown today. Millions of people are infected in the world by this disease.

Applicable prophylactic use of condoms is obviously insufficient.

To prevent this disease medicine should be taken externally with use of condom.

A solution and cream (or a gel), containing active Chlrine in a safe concentration for humans which is capable of quickly killing the virus HIV/AIDS infection on surfaces of the skin and mucous membranes.

Pantocid (Pantosept) is suggested in combination with other necessary substances.

The known use of Pantocid (Pantosept) in tablet form is for disinfection in water, and for irrigation and cleaning of wounds (0.1-0.5 solution).

The solution Pantosept is the suggested use in a mixture with Sodium Tetraborate and hypertonic solution of the Sodium Chloride and Sodium Hydrocarbonate. The mixture hypertonic solution of the Sodium Chloride (4%) will promote the best cleansing from possible presence on mucous membranes and skin of the infected material (mucous, sperm and etc.) due to its osmatic effect. Besides, hypertonic salt solution is a germicidal characteristic.

Hydrocarbonate Sodium is also used for mouthwash, and to treat head colds, conjunctivitis, laringitis, stomatitis, and etc.

Its presence in 5% solutions will promote best dilution Pantosept in water, will increase the osmotic effect of the solution and promote the best cleansing of slime and other infected elements. To increase the antiseptic effect of the solution, add 0.5% sodium tetraborate, which is also known as germicidal preparation and put in practice for mounthwashes. The suggested solution to prevent HIV/AIDS infection is the following: 0.2% Pantocidi (Pantosept), 4% Sodium chloride, 5% Hydrocarbonate Sodium and 0.5-1.0% Sodium tetraborate on distilled water—apply externally of the preventive means solution “B” (Brilman's solution)
Rp: Pantosept 1.0
   Sodium chloride—20.0
   Sodium hydrocarbonate—25.0
   Sodium tetraborate—2.5
   Aq. destill.—500.0
MDS—External (for Stashing, mouthwashes, irrigation)

With this solution is used a partial disinfection processing and also cleaning the skin and mucous membranes including the mouth, sexual organs, anus and crotch, and also any infected material by use of the mouthwashes, washing and irrigation (washing vagina) after contact (sexual relations) with possible virus carrier HIV/AIDS diseases.

After washing with this solution I suggest to apply externally the unguentum (cream or gel) “Oxopant” containing. 0.2% Pantocidi (Pantosept) and 0.5% Oxilini. This medicine is put on the skin and mucous membranes of the sexual organ, anus and crotch for 15-20 min.

The earlier these proposed preventive means are used after sexual relations, the more chances there are for avoiding contamination.

These preparations can be also successfully used for the prevention of gripe and flu viral infection with use of the mouthwash of the throat and drop in nose of the solution “B”0 (Brilman's solution) with following applying “Oxopant” to mucous membranes in the nose.

1. What I claim as my invention is: For preventive maintenance of the contamination viruses HIV/AIDS infection, gripe and flu. I suggest externally applying the solution “B” (Brilman's solution) including Pantocidi (Pantosept)—0.2%. Sodium chloride—4%, Hydrocarbonate Sodium—5% and Sodium Tetraborate—0.5% on distilled water . Subsequently also use externally the proposed unguent (cream or gel)—“Oxopant” inclusive Pantocidi (Pantosept)—0.2% and Oxolini—0.5%
